(19) United States
(12) Patent Application Publication
(10) Pub. No.: US 2024/0366848 A1
(43) Pub. Date: Nov. 7, 2024

(54) APPARATUS AND METHOD FOR TEMPERING SOFT WATER AND/OR PERMEATE FOR A DIALYSIS SYSTEM

(71) Applicant: B. Braun Avitum AG, Melsungen (DE)

(72) Inventors: Philipp Odernheimer, Melsungen (DE); Nino Lieb, Melsungen (DE); Jonas Giesa, Kassel (DE)

(21) Appl. No.: 18/652,598

(22) Filed: May 1, 2024

(30) Foreign Application Priority Data
May 3, 2023 (DE) 10 2023 111 496.4

(51) Int. Cl. A61M 1/16; A61M 1/26; B01D 61/02; B01D 61/12; C02F 1/00; C02F 1/44

(52) U.S. Cl. CPC A61M 1/1664; A61M 1/267; B01D 61/025; B01D 61/12; C02F 1/008; C02F 1/441; A61M 2205/366; B01D 2313/083; B01D 2313/221; B01D 2313/243; C02F 2209/02; C02F 2301/043; C02F 2303/10

(57) ABSTRACT

A device controls temperature of soft water and/or permeate for dialysis applications based on the principle of reverse osmosis. The device, which can be used in a dialysis system, includes a buffer tank for heat which is coupled in terms of heat flow to a heat source and/or heat sink and receives or contains a fluid heat transfer medium. A soft water heat exchanger is connected on the primary side to the buffer tank by a pump circuit for the heat transfer medium, which is connected on the secondary side to the soft water supply line. A permeate heat exchanger is connected on the primary side to the buffer tank by a pump circuit for the heat transfer medium, which is connected on the secondary side to the permeate extraction line.

### CROSS-REFERENCE TO RELATED APPLICATION

This application claims priority under 35 U.S.C. § 119 to German Application No. DE 10 2023 111 496.4, filed on May 3, 2023, the content of which is incorporated by reference herein in its entirety.

### FIELD

The present disclosure relates to a device for controlling the temperature of soft water in a soft water supply line and/or of permeate in a permeate removal line of a water treatment system based on the principle of reverse osmosis and designed for use in a dialysis system. The present disclosure also relates to an associated operating method.

### BACKGROUND

A dialysis system for extracorporeal blood washing requires high-purity water, also known as permeate, which is usually obtained from softened water, so-called soft water, in a water treatment system using the principle of reverse osmosis. Softened means that substances responsible for the calcification of pipes have been removed from the soft water. The water treatment system can be an integral part or an associated component of the dialysis system and has a soft water supply line and a permeate removal line, also known as permeate discharge line. The permeate removal line typically leads to a ring line to which mobile dialysis machines can be connected as consumers of permeate.

For energy-efficient operation, it is desirable to centrally temper or temperature-condition (preheat or cool) both the soft water in the soft water supply line and the permeate in the permeate extraction line, which is correspondingly energy-intensive. The heating or cooling capacity to be provided for peak loads must be greater than that required for continuous extraction, especially in the case of strongly fluctuating permeate extraction. With integrated hot cleaning in particular, it may be necessary to heat up the system considerably beyond normal operation.

Current systems for influencing the permeate temperature in the ring line are limited to the purely electrical heating of permeate for thermal disinfection. Centralized preheating of the permeate during dialysis operation is not provided. Central cooling of the permeate is also not possible. The current systems do not interact with the soft water and therefore cannot temperature-condition it.

### SUMMARY

The object of the present disclosure is to provide a device of the type described above which enables reliable temperature control of soft water and/or permeate for dialysis applications, which can be flexibly adapted to changing requirements, at low operating costs. Furthermore, a particularly advantageous operating method for such a device is to be disclosed.

Accordingly, a device is provided for controlling the temperature of soft water in a soft water supply line and/or of permeate in a permeate removal line of a water treatment system based on the principle of reverse osmosis and designed for use in a dialysis system, comprising a buffer storage tank for heat which is or can be coupled in terms of heat flow to a heat source and/or heat sink and which receives or contains a fluid working medium (also called heat transfer medium or buffer medium or buffer storage medium) as heat transfer medium, and
a soft water heat exchanger connected on the primary side to the buffer tank by means of a pump circuit for the heat transfer medium, which is connected on the secondary side to the soft water supply line, and
a permeate heat exchanger connected to the buffer tank on the primary side by means of a pump circuit for the heat transfer medium, which is connected to the permeate extraction line on the secondary side.

The use of a thermal buffer tank, which is thermally coupled or can be coupled to both the soft water supply line and the permeate extraction line of the water treatment system, and which in turn is coupled or can be coupled to an external heat source and/or heat sink in terms of heat flow, makes it possible to reduce operating costs by using environmental energy or other systems for cost-effective heat supply or heat removal. The system is especially designed for highly fluctuating permeate consumption and the associated fluctuations in thermal energy requirements. By combining a thermal energy storage unit designed for buffering and by adjusting the convection flows on the primary sides of the heat exchangers, a fluctuating thermal energy requirement can be balanced out. Modularity allows the system to be adapted to and integrated into a wide range of dialysis systems, depending on the specific requirements and installation situation.

Advantageous embodiments are the subject of the following description.

In an advantageous embodiment, the heat source and/or heat sink is a heat pump, preferably an air-to-water heat pump. An air-to-water heat pump uses the ambient air as a heat source. During operation, a fan actively draws in the air and passes it on to a heat exchanger, the evaporator. A refrigerant circulates in this, which changes its state of aggregation even at low temperatures due to its thermal properties. When the refrigerant exchanges heat with the incoming outside air, it heats up until it finally evaporates. A downstream, electrically driven compressor uses electrical energy to increase the pressure and temperature of the refrigerant vapor. Once the refrigerant vapor has reached the desired temperature level, it flows on to the next heat exchanger, the condenser. Here it transfers its heat to a liquid working medium, usually water, and condenses. The water heated in this way is now fed into the storage tank of the buffer tank within the context of the present disclosure, typically by a pump, and is available there as a fluid working medium or heat transfer medium in order to transfer the stored thermal energy to the soft water and/or the permeate as required.

The working medium used in the context of the present disclosure is preferably water, possibly with additives, or an aqueous solution, but other media with suitable thermal properties are also possible.

The working medium within the buffer storage tank (also: buffer storage medium) is preferably designed or provided in such a way that detection of the working medium in the permeate is possible via electrical or optical measurements, in order to detect a leakage (in particular within the permeate heat exchanger) into the permeate line(s) and an associated contamination of the permeate in this way. Advantageously, the device according to the present disclosure is therefore equipped with suitable detectors which can issue a corresponding warning via the system control and, if necessary, interrupt the permeate discharge.

In other words, the working or buffer medium is advantageously designed in such a way that a substance contained in it (ideally biologically harmless) can be detected by a sensor. Example: The buffer medium is heavily mixed with Na+ ions. As soon as the Na+-containing buffer medium enters the permeate, it can be detected ion-specifically/ion-unspecifically via an ammeter or a conductivity measuring electrode. Alternatively or additionally, a dye and/or an optically active substance could be added to the buffer medium so that the substance can be detected optically when it enters the permeate.

Preferably, the working medium is transported from the heat pump to the buffer tank and back in a circuit driven by one or more pumps, whereby passive convection transport is also or alternatively possible.

Advantageously, the heat pump can be reversed in its circulation direction and can therefore also be used as a chiller or heat sink in order to cool the working medium—and thus the soft water and/or the permeate—if required.

In an advantageous variant, several heat pumps are connected in parallel to the buffer storage tank as a heat source and/or heat sink in order to increase the maximum heating or cooling capacity and to develop different types of heating and/or cooling reservoirs in accordance with the principles of redundancy and/or diversity.

For a particularly compact solution, the soft water heat exchanger and/or the permeate heat exchanger can be structurally integrated into the buffer cylinder.

The heat exchangers used in the system, i.e. in particular the soft water heat exchanger and/or the permeate heat exchanger, can advantageously be designed as tubular coil heat exchangers for efficient heat transfer. Such tubular coil heat exchangers can be designed in particular as corrugated tube heat exchangers.

Advantageously, a bypass line bypassing the soft water heat exchanger is connected to the soft water supply line. Similarly, a bypass line bypassing the permeate heat exchanger is advantageously connected to the permeate extraction line. The temperature of the soft water or permeate can be set easily, quickly and to a certain extent independently of the temperature of the working medium in the buffer tank by appropriately adjusting the mixing ratio of the heat exchanger flow and bypass flow using control valves or similar. As an alternative or in addition to the valves, pumps can also be used to control or regulate the mixture of tempered (passing through the respective heat exchanger) and non-tempered (passing through the respective bypass) permeate/soft water.

Advantageously, the respective pump circuit has a variable-speed pump for the working medium in order to be able to adjust or regulate the heat supply or removal provided on the primary side in the soft water heat exchanger and/or in the permeate heat exchanger as required.

In a preferred variant, an additional heater is connected in the permeate extraction line to heat the permeate to a sufficient disinfection temperature for thermal disinfection of subsequent line sections. This additional heater is preferably an electric heater. The additional heater is preferably arranged downstream of the permeate heat exchanger in relation to the permeate. By preheating the permeate with the aid of the buffer tank, the auxiliary heater only has to heat the remaining temperature difference between the temperature of the permeate at the outlet of the permeate heat exchanger and the desired disinfection temperature in the ring line.

According to the present disclosure, the heat transfer medium from the buffer tank (and not a separate working medium) is circulated in the respective pump circuit. This means that the heat transfer medium is taken from the buffer tank, fed via the respective pump circuit to the soft water heat exchanger/permeate heat exchanger and from there returned to the buffer tank. In this respect, each pump circuit is a pump circuit for heat transfer medium from the buffer tank. The heat transfer medium from the buffer cylinder therefore flows through the soft water heat exchanger/permeate heat exchanger on the primary side.

The present disclosure also relates to a dialysis system with a water treatment system based on the principle of reverse osmosis, which has a soft water supply line and a permeate removal line, and which is equipped with or can be connected to a device of the type described above, namely for tempering soft water in the soft water supply line and/or permeate in the permeate removal line.

When operating the device described above, it is advantageous if the heat flow from the heat source to the buffer tank or from the buffer tank to the heat sink is controlled as a function of a temperature of the working medium measured in the buffer tank. More precisely, the decision as to whether the heat pump serves as a sink or source is preferably made via a temperature sensor at the soft water inlet (before flowing through the soft water heat exchanger), i.e. depends on the inlet temperature of the soft water. The subsequent control of the heat pump is then carried out, as mentioned above, using the temperature of the working medium in the buffer tank.

In an embodiment which is considered to be inventive in its own right and independent of the method described in the last paragraph, the pump circuit with the soft water heat exchanger has a variable-speed pump for the working medium on the primary side, the speed of which is controlled as a function of a soft water temperature measured downstream of the soft water heat exchanger on the secondary side.

In an embodiment that is also considered to be inventive and independent, the pump circuit with the permeate heat exchanger has a variable-speed pump for the working medium on the primary side, the speed of which is controlled as a function of a permeate temperature measured downstream of the permeate heat exchanger on the secondary side.

In one variant, which is particularly advantageous in the case of the above-mentioned auxiliary heating, the permeate is heated to a disinfection temperature during a disinfection process and, once a pipe section has been disinfected, is passed (back) through the permeate heat exchanger, whereby the permeate releases any residual heat to the buffer tank. The recovered heat is then available again for various applications.

In an advantageous embodiment of this cooling after hot disinfection, a heat pump with a compressor, a heat exchanger and a fan associated with the heat exchanger is provided as a heat sink, whereby the (re) cooling of the buffer tank after disinfection is carried out or supported actively or passively by the heat pump. In the active variant, the heat pump participates in the cooling process while the compressor is running; in the passive variant, only the heat exchanger of the heat pump (compressor switched off/fan switched on) is used to dissipate heat to the environment.

In summary, the present disclosure comprises a device and a method for coupling a permeate and/or soft water line of a water treatment plant via a heat exchanger in each case with a buffer tank of a centralized or decentralized heat generator for heating or cooling the fluids within the lines. A modular system is thus realized for centralized:
temperature control or temperature conditioning (preheating/cooling) of permeate during dialysis treatment,
thermal disinfection of at least one permeate ring line and connected dialysis machine & reverse osmosis system, and/or
tempering or temperature conditioning (preheating/cooling) of soft water before it enters the reverse osmosis system.

### BRIEF DESCRIPTION OF THE DRAWINGS

Several embodiments of the present disclosure are explained below with reference to the accompanying drawings.

FIG. 1 shows a schematic representation of a device for controlling the temperature of soft water in a soft water supply line and/or of permeate in a permeate extraction line of a water treatment system based on the principle of reverse osmosis and designed for use in a dialysis system,

FIG. 2 shows a modification of the device of FIG. 1; and

FIG. 3 shows a further modification of the device of FIG. 1.

### DETAILED DESCRIPTION

Elements that are identical or have the same effect are marked with the same reference symbols in all figures.

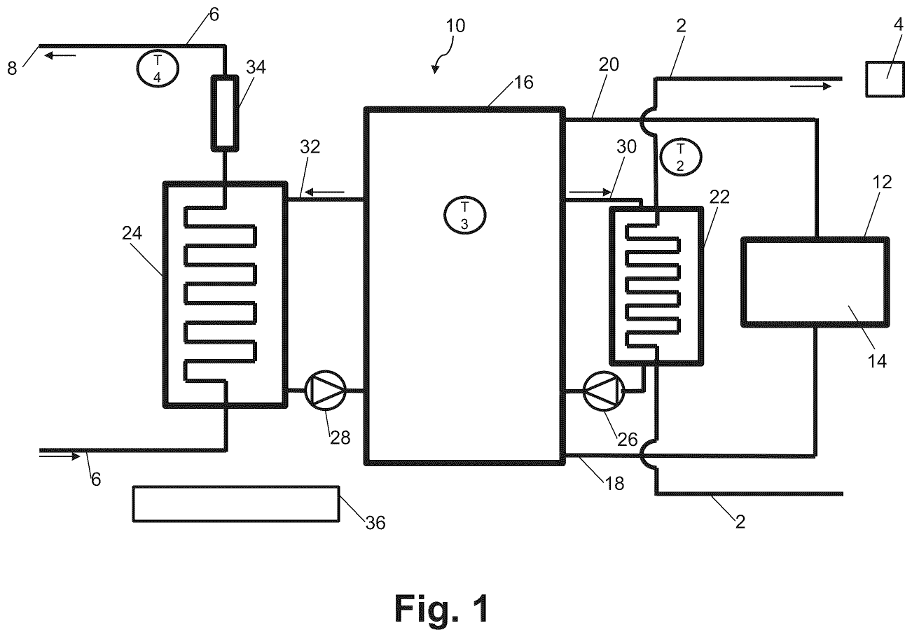

FIG. 1 schematically shows the device and process diagram of the preferred embodiment. Softened water, called soft water, is supplied via a soft water supply line 2 to a water treatment plant 4, which is only indicated schematically, where it is treated and purified for dialysis applications according to the principle of reverse osmosis. The high-purity water, known as permeate, leaves the water treatment plant 4 via a permeate extraction line 6 and, in the example, is fed via this to a permeate ring line 8, to which mobile dialysis machines can be connected as consumers of permeate.

The soft water coming from the upstream softening stage (not shown) at a temperature of 15° C., for example, must be heated to a temperature of 25° C., for example, for efficient reverse osmosis. Similarly, the permeate leaving the water treatment plant 4 at a temperature of, for example, 25° C. (corresponding to the soft water temperature there) must be heated to a temperature of, for example, 36° C. (body temperature) before being used as dialysis fluid. In other scenarios, it may be necessary to cool the fluids. For this purpose, a device 10 for tempering soft water and/or permeate is provided, which is described in more detail below.

From a heat generator acting as a heat source 12, in the preferred embodiment a heat pump 14, in particular an air-to-water heat pump, the thermal energy (Q_{zu}) extracted from the environment is supplied to the heat or buffer tank 16 in the form of heat. The buffer tank 16 comprises a thermally insulated container for a fluid storage or working medium which acts as a heat transfer medium, essentially water in this example. In this example, the buffer tank 16 and the heat pump 14 are components of a closed circuit for the working medium: comparatively cold working medium is removed from the buffer tank 16 via the cold medium line 18, passed through a heating section containing the heat pump 14, and finally fed back into the buffer tank 16 via the hot medium line 20. To transport the working medium, a circulation pump (not shown) can be connected to the circuit, possibly realized by the heat pump 16 itself. In the embodiment example, the setpoint or target temperature of the working medium in the buffer tank 16 is approximately 40° C.

Two heat exchangers 22, 24 are connected to the buffer tank 16 on the primary side via pipelines. Variable-speed pumps 26, 28 are arranged in the pipelines. This means that two pump circuits 30, 32 are realized on the primary side for the working medium, namely from the buffer tank 16 via the respective heat exchanger 22, 24 and back into the buffer tank 16. As already mentioned, the soft water heat exchanger 22 connected on the secondary side in the soft water supply line 2 is used to heat the soft water, which is subsequently fed into the reverse osmosis system realized within the water treatment plant 4. By raising the soft water inlet temperature, the specific energy requirement, characterized by the energy consumption of the osmosis pump, decreases during operation of the reverse osmosis system. The permeate heat exchanger 24 connected on the secondary side in the permeate extraction line 6, on the other hand, is used to heat the permeate to a set temperature during dialysis treatment before it enters the ring line 8 or the dialysis machines.

During thermal disinfection of the ring line 8 by hot permeate, the storage temperature, i.e. the temperature of the working medium in the buffer tank 16, can be increased to a technically sensible level by the heat generator or the heat pump 14. Reheating to the required target temperature is achieved by an additional heater 34, in the preferred embodiment an electric auxiliary heater. For this purpose, the additional heater 34 is connected downstream of the permeate heat exchanger 24 in the permeate extraction line 6. Once disinfection has been completed, some of the previously supplied energy can be transferred from the permeate through the permeate heat exchanger 24 to the storage or working medium of the energy or buffer tank 16. The recovered energy can then be used to preheat the soft water and/or the permeate.

In very warm regions, in which the soft water temperature on the inlet side is above a defined limit value, energy can be extracted from the heat or buffer tank 16 and released to the environment (Q_{ab}) by reversing the circulation of the heat pump 14, whereby the soft water and/or the permeate are cooled as they flow through the heat exchangers 22, 24. This can reduce the water requirement as a result of the temperature drop in the reverse osmosis system.

The control and regulation of the individual actuators can be carried out by a central control unit 36, which is only schematically indicated here, or can be implemented decentrally. A preferred control of the individual actuators is listed below, taking into account the respective process variables:
The heat generator or heat pump 14 is controlled by recording and evaluating measurement data from the temperature sensor T3, which measures the temperature of the storage medium in the buffer tank 16.
The pump 26 in the pump circuit 30 for the soft water temperature control is controlled by recording and evaluating measurement data from the temperature sensor T2. The temperature sensor T2 is connected downstream of the soft water heat exchanger 22, e.g. at its outlet, in the soft water supply line 2.
The pump 28 in the pump circuit 32 for the permeate temperature control is controlled by recording and evaluating measurement data from the temperature sensor T4. The temperature sensor T4 is connected downstream of the permeate heat exchanger 24, preferably downstream of the additional heating section with the additional heater 34, in the permeate extraction line 6.
The additional heating section, i.e. the additional heater 34, is controlled by recording and evaluating measurement data from the temperature sensor T4 located at the output of the additional heating section or further downstream.

The device 10 shown in FIG. 1 can undergo a number of modifications which are within the scope of the present disclosure, and which can be combined with one another as desired, insofar as this makes technical sense. In particular, the following modifications can be realized:
The required heat can also be provided by another type of decentralized or centralized heat generator instead of the heat pump 14.
The required heat provided by the additional electric heater 34 can also be provided by another type of decentralized or centralized heat generator.
Thanks to the modular design, the system can also be designed without a soft water heat exchanger 22 or permeate heat exchanger 24.
If thermal disinfection is not required, the additional heater 34 can be omitted.
If the heat generator or heat pump 14 is technically capable of providing the required disinfection temperature, an additional heater 34 is not required.

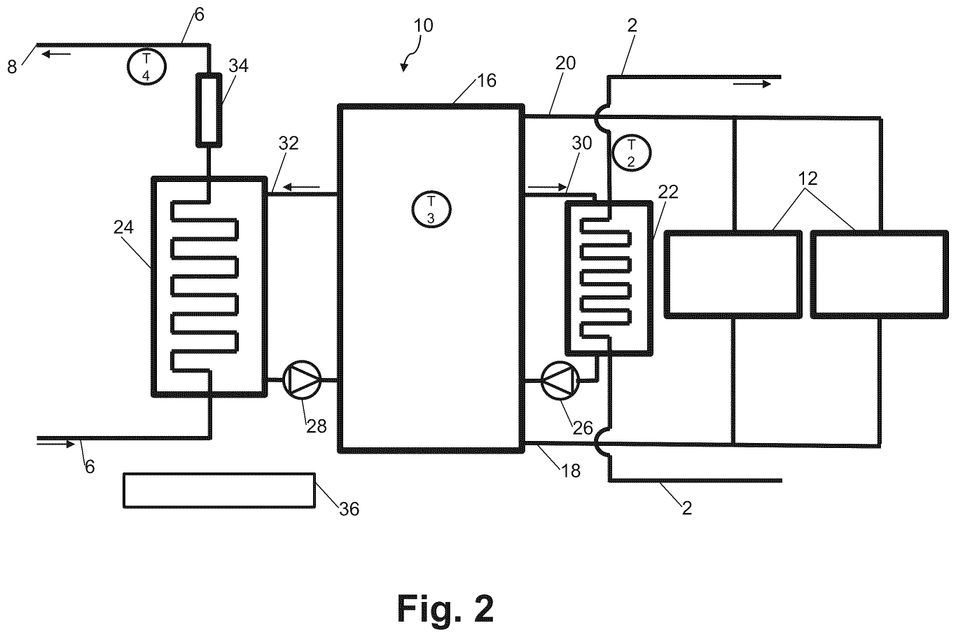

For higher heat requirements, several heat generators, in particular heat pumps 14, can be connected in parallel to the heat or buffer tank 16 as shown in FIG. 2. This variant can be combined with all others.

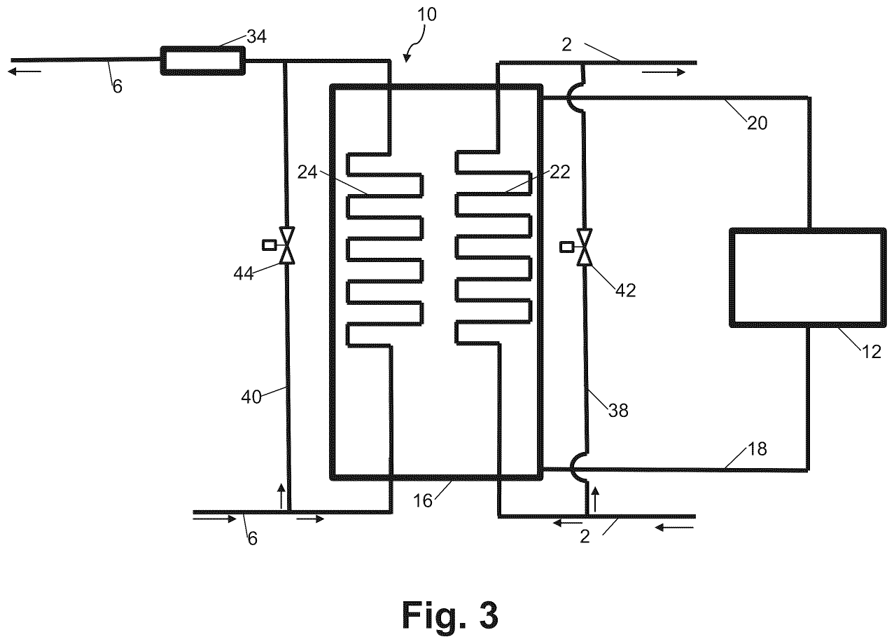

In another embodiment according to FIG. 3, the heat exchangers 22, 24 are integrated into the buffer tank 16. The primary-side pump circuits 30, 32 with the pumps 26, 28 can be omitted here because the heat exchangers (e.g. corrugated pipes) are already integrated in the storage medium (heat transfer medium). The heating section with the additional heater 34 can also be optionally integrated into the system. These variants can also be combined with all others.

To prevent an impermissible temperature increase in the event of low water consumption (permeate and/or soft water) and an increased storage temperature, colder water can be mixed in via the bypasses 38, 40 as shown in FIG. 3 in order to maintain the required target temperature. The valves 42, 44 required for this in the line system can be designed as multi-way valves (at the junctions or line branches or line junctions), control valves or solenoid valves, for example. These variants can also be combined with all the other variants described above.

### LIST OF REFERENCE SYMBOLS

2 Soft water supply line
4 Water treatment plant
6 Permeate extraction line
8 Ring line
10 Device
12 Heat source
14 Heat pump
16 Buffer tank
18 Cold medium line
20 Hot medium line
22 Soft water heat exchanger
24 Permeate heat exchanger
26 Pump
28 Pump
30 Pump circuit
32 Pump circuit
34 Additional heater
36 Control unit
38 Bypass line
40 Bypass line
42 Valve
44 Valve
T2 . . . T4 Temperature sensors/Temperatures

1. A device for controlling temperature of soft water in a soft water supply line and/or of permeate in a permeate extraction line of a water treatment plant that operates based on reverse osmosis and is designed for use in a dialysis plant, the device comprising:
a buffer tank for heat which is coupled in terms of heat flow to a heat source and/or heat sink and receives or contains a fluid heat transfer medium;
a soft water heat exchanger connected on a primary side to the buffer tank by a pump circuit for the fluid heat transfer medium, which is connected on a secondary side to the soft water supply line; and
a permeate heat exchanger connected on a primary side to the buffer tank by a pump circuit for the heat transfer medium, the permeate heat exchanger connected on a secondary side to the permeate extraction line.

2. The device according to claim 1, wherein the heat source and/or heat sink is a heat pump.

3. The device according to claim 2, wherein the heat pump is reversible in a circuit direction.

4. The device according to claim 3, further comprising a control or regulating device that adjusts the circuit direction based on a soft water inlet temperature measured in the soft water supply line upstream of the soft water heat exchanger.

5. The device according to claim 2, wherein the heat pump comprises a plurality of heat pumps connected in parallel as the heat source and/or heat sink.

6. The device according to claim 1, wherein the soft water heat exchanger and/or the permeate heat exchanger is/are structurally integrated into the buffer tank.

7. The device according to claim 1, wherein a bypass line bypassing the soft water heat exchanger is connected into the soft water supply line, and/or wherein a bypass line bypassing the permeate heat exchanger is connected into the permeate extraction line.

8. The device according to claim 1, wherein the respective pump circuit has a variable-speed pump for the heat transfer medium.

9. The device according to claim 1, wherein an additional heater for heating the permeate to a disinfection temperature sufficient for thermal disinfection of subsequent line sections is connected into the permeate extraction line.

10. The device according to claim 9, wherein the additional heater is an electric heater.

11. The device according to claim 9, wherein the additional heater is arranged downstream of the permeate heat exchanger with respect to the permeate.

12. The device according to claim 1, wherein the device is configured to:
control heat flow from the heat source to the buffer tank; or
control heat flow from the buffer tank to the heat sink as a function of a temperature of the heat transfer medium measured in the buffer tank.

13. The device according to claim 12, wherein the pump circuit with the soft water heat exchanger has a pump for the heat transfer medium on the primary side, a speed of said pump being controlled as a function of a soft water temperature measured on the secondary side downstream of the soft water heat exchanger.

14. The device according to claim 12, wherein the pump circuit with the permeate heat exchanger has a pump for the heat transfer medium on the primary side, a speed of said pump being controlled as a function of a permeate temperature measured on the secondary side downstream of the permeate heat exchanger.

15. The device according to claim 12, wherein the device is configured to heat the permeate to a disinfection temperature and, after disinfection of a line section has been completed, passes the permeate through the permeate heat exchanger and releases any residual heat present to the buffer tank.

16. The device according to claim 15, further comprising a heat pump with a compressor and a fan that acts a heat sink, wherein the heat pump is operated as the heat sink during or after cooling of the permeate.

17. A dialysis plant with a water treatment plant operating based on reverse osmosis, the dialysis plant comprising:
the device according to claim 1;
a soft water supply line; and
a permeate extraction line,
the device configured for tempering soft water in the soft water supply line and/or permeate in the permeate extraction line.
